(19) United States
(12) Patent Application Publication
(10) Pub. No.: US 2010/0184706 A1
(43) Pub. Date: Jul. 22, 2010

(54) FAP-ACTIVATED CHEMOTHERAPEUTIC COMPOUNDS, AND METHODS OF USE THEREOF

(76) Inventors: William W. Bachovchin, Cambridge, MA (US); Hung-Sen Lai, Andover, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/531,529

(22) PCT Filed: Mar. 20, 2008
(86) PCT No.: PCT/US08/57633

§ 371 Date: Mar. 29, 2010

Related U.S. Application Data
(60) Provisional application No. 60/895,786, filed on Mar. 20, 2007.

(51) Int. Cl. A61K 38/05; C07K 2/00; C07K 5/06; C07K 5/08; C07K 5/10; A61K 38/06; A61K 38/07; C12N 5/09; A61P 35/04

(52) U.S. Cl. 514/18; 530/300; 562/553; 530/331; 530/330; 548/572; 514/2; 514/19; 435/375

(57) ABSTRACT

One aspect of the present invention relates to compounds for delivering chemotherapeutic agents to cancerous cells to reduce discomfort and deleterious side effects associated with systemic administration of such agents. Another aspect of the invention relates to methods of treating a subject with cancer by administering a therapeutically effective amount of a chemotherapeutic agent that predominately targets cancerous cells over healthy cells. Additionally, one aspect of the present invention relates to methods of inhibiting the life-cycle of a cancerous cell, as well preparing a chemotherapeutic prodrug.

### RELATED APPLICATIONS

This application claims the benefit of priority to U.S. Provisional Patent Application Ser. No. 60/895,786, filed Mar. 20, 2007.

### BACKGROUND OF THE INVENTION

Fibroblast activation protein α (FAP) is a protease expressed on reactive stromal fibroblasts surrounding newly formed blood vessels in greater than 90% of common human epithelial cancers, in granulation of healing wounds, in cirrhotic human liver cells, and in bone and soft tissue sarcomas. FAP has been implicated in extracellular matrix remodeling, tumor growth, and metastasis. Studies have suggested that FAP inhibition may attenuate tumor growth, making this protease a potential therapeutic target.

FAP is a type II transmembrane serine protease (seprase) belonging to the prolyl oligopeptidase family. It has both in vitro dipeptidyl peptidase activity, meaning that it is capable of cleaving N-terminal dipeptides from polypeptides, and collagenolytic activity, meaning that it is capable of degrading gelatin and type I collagen. Interestingly, both functions utilize a common active site in FAP. However, the enzyme's precise in vivo action and specific roles in tumor growth and invasion remain elusive. Moreover, the exact molecular mechanisms the enzyme utilizes remain largely unknown, mainly because inhibitors that distinguish FAP from other prolyl peptidases, like dipeptidyl peptidase-IV (DPP-IV), have been developed only recently.

Indeed, DPP-IV is the closest homolog of FAP, sharing approximately 50% sequence identity. Aertgeerts et al. (2005) J. Biol. Chem. 280(20):19441 showed that, like the extracellular fold portion exhibited by DPP-IV, each FAP subunit features a topologically distinct eight-bladed N-terminal β-propeller domain and a C-terminal α/β-hydrolase domain. The β-propeller has several potential sites of N-linked glycosylation and follows a 20-amino acid transmembrane domain and six-amino acid cytoplasmic tail. Crystallographic data for both FAP and DPP-IV suggest that the β-propeller domain and the α/β-hydrolase domain contain important substrate-binding sites and that key substrate-binding residues in both proteases are in similar positions. However, in contrast to DPP-IV, an Ala reside (A⁶⁵⁷) located near the S₂ pocket in FAP allows it to function as both a dipeptidyl peptidase and an endopeptidase.

The full spectrum of FAP's endopeptidase specificity has not been elucidated. Recently, efforts have been made to determine said specificity more narrowly, and subsequently to identify peptide motifs in order to exploit those characteristics toward FAP-selective inhibitor design. For example, Edosada et al. (2006) FEBS Letters 580(6):1581 synthesized intramolecularly-quenched fluorescent substrate sets based on the FAP cleavage site in α₂-antiplasmin (TSGP-NQ) to elucidate further FAP P₄-P′₂ specificity. This analysis determined that FAP requires substrates with Pro at P₁; Gly, D-Ala, or D-Ser at P₂; and that FAP prefers small, uncharged amino acids at P₃, while tolerating most amino acids at P₄, P′₁, and P′₂. These substrate preferences allowed for the design of peptidyl-chloromethyl ketones that inhibited FAP, but not DPP-IV.

Thus, FAP contains a well-defined, hydrophobic S₁ binding pocket that best accommodates substrates with a P₁ Pro residue. Edosada et al. (ibid.) go further in stating that, “Beyond P₁, [their] model predicts that substrates must contain a small amino acid able to adopt a positive phi value in the Ramachandran plot to avoid steric clashes [with] the protease,” which explains their observations of FAP activity described above. However, based on their structural analysis of the FAP active site and FAP substrate binding sites, Aertgeerts et al. (ibid.) state that, “The S₁′ subsite in FAP[α] is flat and could accommodate most amino acids.”

Edosada et al. (2006) J. Biol. Chem. 281(11):7437 have also shown that FAP cleaves N-terminus formyl-, benzyloxycarbonyl-(Z), biotinyl, and peptidyl-Gly-Pro substrates, which DPP-IV cleaves poorly, suggesting that an N-Acyl-Xaa-Pro motif might be advantageous for inhibitor design. Accordingly, they identified Ac-Gly-boroPro as a FAP-selective inhibitor and suggested that N-Acyl-Gly-Pro-based inhibitors will allow testing of FAP as a therapeutic target.

In general, chemotherapeutic agents are toxic substances that do not discriminate well between healthy and cancerous cells. Therefore, systemic administration of chemotherapeutic agents often results in great discomfort and deleterious side effects for a cancer patient. The list of discomforts and side effects is extensive and includes nausea, diarrhea, anemia, hair loss, depression of the immune system, hemorrhaging, secondary neoplasms, cardiotoxicity, hepatoxicity, nephrotoxicity, and ototoxicity. It would be beneficial to a patient in need of such treatments to be able to target preferentially the cancerous cells, leaving healthy cells relatively undamaged. Importantly, chemotherapeutic agents that are bound to an oligopeptide sequence that is a portion or all of a substrate for an activating protease will preferentially target cancerous cells that overexpress the activating protease. FAP is an example of an activating protease that is often overexpressed in certain cancer cells.

### SUMMARY OF THE INVENTION

It is an object of the present invention to provide compounds for delivering chemotherapeutic agents to cancerous cells, thereby reducing the discomfort and deleterious side effects associated with systemic administration.

It is another object of the present invention to provide a method of treating a subject with cancer by administering to the subject a therapeutically effective amount of a chemotherapeutic agent that predominately targets cancerous cells over healthy cells.

It is another object of the present invention to provide a method of inhibiting the life-cycle of a cancerous cell.

It is another object of the present invention to prepare a chemotherapeutic prodrug.

In one embodiment, the present invention relates to a compound represented by formula 1:

A-(B)ₙ—C 1
or a pharmaceutically acceptable salt or composition thereof;
wherein
A represents an oligopeptide sequence that is a portion or all of a substrate for an activating protease, wherein said oligopeptide sequence comprises a bond cleavable by said activating protease;
B is independently for each occurrence a D-isomer or L-isomer of a naturally occurring alpha-amino acid;
C represents a chemotherapeutic agent; and
n is an integer in the range of zero to about five.

In another embodiment, the present invention relates to a method of treating cancer, comprising administering to a subject in need thereof a therapeutically effective amount any of the aforementioned compounds. In a further embodiment, the subject is a primate, bovine, ovine, equine, porcine, rodent, feline, or canine. In a further embodiment, the subject is a human. In a further embodiment, the subject may be treated for breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, or adenocarcinoma.

In another embodiment, the present invention relates to a method of inhibiting the life-cycle of a cancer cell comprising: contacting the cancer cell with a chemotherapeutic agent bound to an oligopeptide sequence that is a portion or all of a substrate for an activating protease, wherein said oligopeptide sequence comprises a bond cleavable by said activating protease.

In another embodiment, the present invention relates to a method of preparing a chemotherapeutic prodrug comprising binding a chemotherapeutic agent to an oligopeptide sequence that is a portion or all of a substrate for an activating protease, wherein said oligopeptide sequence comprises a bond cleavable by said activating protease.

In the above embodiments, the activating protease may be a serine protease, and may also be one selected from the group consisting of fibroblast activation protein (FAP), dipeptidyl-peptidase IV, acylaminoacyl-peptidase, lysosomal Pro-Xaa carboxypeptidase, nucleoporin, lactoferrin, glycosylasparaginase precursor, calpain-2, poliovirus-type picornain 3C, poliovirus-type picornain 2A, parechovirus picornain 3C, adenain, ubiquitinyl hydrolase-L1, ubiquitin-specific peptidase 14, amidophosphoribosyltransferase precursor, autophagin-1, Cezanne deubiquitinating peptidase, otubain-1, CylD protein, UL36 deubiquitinylating peptidase, pepsin A, HIV-1 retropepsin, spumapepsin, presenilin 1, impas 1 peptidase, aminopeptidase N, angiotensin-converting enzyme peptidase unit 1, matrix metallopeptidase-1, neprilysin, carboxypeptidase A1, eupitrilysin, membrane dipeptidase, pappalysin-1, S2P peptidase, AMSH deubiquitinating peptidase, and scytalidoglutamic peptidase. In one embodiment, the activating protease is FAP. In a further embodiment, the oligopeptide comprises at least two, three, or four amino acids. The oligopeptide is a substrate of the activating protease comprising a bond cleavable by the activating protease. In a further embodiment, the cleavable bond is carboxy terminal to a dipeptide sequence or subsequence selected from the group consisting of Gly-Pro; Ala-Pro; Trp-Pro; (acetylated)Gly-Pro; (acetylated)Ala-Pro; and (acetylated)Trp-Pro.

The chemotherapeutic agent, C, may in one embodiment, be bound to the C-terminus of the A-(B)ₙ oligopeptide sequence. In a further embodiment, C is an alkylating agent, antimetabolite, anthracycline, plant alkaloid, topoisomerase inhibitors, or an antitumor antibiotic.

Examples of alkylating agents include cyclophosphamide, chlorambucil, estramustine, melphalan, mechlorethamine, ifosfamide, busulfn, treosulfan, thioTEPA, lomustine, carmustine, streptozocin, temozolomide, dacarbazine, cisplatin, carboplatin, oxaliplatin, procarbazine, trofosfamide, and uramustine.

Examples of antimetabolites include methotrexate, pemetrexed, fludarabine cytarabine, 5-fluorouracil, floxuridine, gemcitabine, trimetrexate, mycophenolic acid, tiazofurin, ribavirin, EICAR, doxifluridine, ratitrexed, cytosine arabinoside, mercaptopurine, thioquanine, and capecitabine.

Examples of plant alkaloids include vinblastine, vincristine, vinorelbine, vindesine, etoposide, teniposide, paclitaxel, and docetaxel.

Examples of topoisomerase inhibitors include amsacrin, etoposide, teniposide, 9-aminocamptothecin, crisnatol, topotecan and irinotecan.

Examples of antitumor antibiotics include actinomycin, dactinomycin, doxorubicin, pirarubicin, zorubicin, daunorubicin, epirubicin, idarubicin, mitoxantrone, bleomycin A2, bleomycin B2, peplomycin, mitomycin C, plicamycin, deferoxamine, and hydroxyurea.

These embodiments of the present invention, other embodiments, and their features and characteristics, will be apparent from the description, drawings and claims that follow.

### BRIEF DESCRIPTION OF THE FIGURES

FIG. 1 depicts as a function of the D-amino acid in the X position the fraction cleaved in vitro by FAP of the individual members of a library of C-terminal gem-amino modified P₂-P′₄ hexapeptide derivatives [XP-YSWS(NH₂)], wherein the N-termini are not protected.

FIG. 2 depicts as a function of the D-amino acid in the X position the fraction cleaved in vitro by FAP of the individual members of a library of C-terminal gem-amino modified P₂-P′₄ hexapeptide derivatives [XP-YSWS(NH₂)], wherein the N-termini are acetylated.

FIG. 3 depicts a general scheme for FAP-activated proteasome inhibition by oxocarbonyl compounds of the present invention.

FIG. 4 depicts a general scheme for FAP-activated proteasome inhibition by thiocarbonyl compounds of the present invention.

### DETAILED DESCRIPTION OF THE INVENTION

### Definitions

For convenience, certain terms employed in the specification, examples, and appended claims are collected here.

The term “amino acid” is intended to embrace all compounds, whether natural or synthetic, which include both an amino functionality and an acid functionality, including amino acid analogues and derivatives. In certain embodiments, the amino acids contemplated in the present invention are those naturally occurring amino acids found in proteins, or the naturally occurring anabolic or catabolic products of such amino acids, which contain amino and carboxyl groups.

Naturally occurring amino acids are identified throughout by the conventional three-letter and/or one-letter abbreviations, corresponding to the trivial name of the amino acid, in accordance with the following list. The abbreviations are accepted in the peptide art and are recommended by the IUPAC-IUB commission in biochemical nomenclature.

| Amino Acid | Three-letter | One-letter |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Unknown or “other” | Xaa | X |

The term “amino acid residue” further includes analogues, derivatives, and congeners of any specific amino acid referred to herein, as well as C-terminal or N-terminal protected amino acid derivatives (e.g., modified with an N-terminal or C-terminal protecting group).

The term “peptide,” as used herein, refers to a sequence of amino acid residues linked together by peptide bonds or by modified peptide bonds. The term “peptide” is intended to encompass peptide analogues, peptide derivatives, peptidomimetics and peptide variants. The term “peptide” is understood to include peptides of any length. Peptide sequences set out herein are written according to the generally accepted convention whereby the N-terminal amino acid is on the left, and the C-terminal amino acid is on the right.

The term “peptide analogue,” as used herein, refers to a peptide comprising one or more non-naturally occurring amino acid. Examples of non-naturally occurring amino acids include, but are not limited to, D-amino acids (i.e., an amino acid of an opposite chirality to the naturally occurring form), N-α-methyl amino acids, C-α-methyl amino acids, β-methyl amino acids, β-alanine ((β-Ala), norvaline (Nva), norleucine (Nle), 4-aminobutyric acid (γ-Abu), 2-aminoisobutyric acid (Aib), 6-aminohexanoic acid (ε-Ahx), ornithine (orn), hydroxyproline (Hyp), sarcosine, citrulline, cysteic acid, cyclohexylalanine, α-amino isobutyric acid, t-butylglycine, t-butylalanine, 3-aminopropionic acid, 2,3-diaminopropionic acid (2,3-diaP), D- or L-phenylglycine, D- or L-2-naphthylalanine (2-Nal), 1,2,3,4-tetrahydroisoquinoline-3-carboxylic acid (Tic), D- or L-2-thienylalanine (Thi), D- or L-3-thienylalanine, D- or L-1-, 2-, 3- or 4-pyrenylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine, D- or L-p-methoxybiphenylalanine, methionine sulphoxide (MSO) and homoarginine (Har). Other examples include D- or L-2-indole(alkyl)alanines and D- or L-alkylalanines, wherein alkyl is substituted or unsubstituted methyl, ethyl, propyl, hexyl, butyl, pentyl, isopropyl, iso-butyl, or iso-pentyl, and phosphono- or sulfated (e.g., —SO₃H) non-carboxylate amino acids.

Other examples of non-naturally occurring amino acids include 3-(2-chlorophenyl)-alanine, 3-chloro-phenylalanine, 4-chloro-phenylalanine, 2-fluoro-phenylalanine, 3-fluoro-phenylalanine, 4-fluoro-phenylalanine, 2-bromo-phenylalanine, 3-bromo-phenylalanine, 4-bromo-phenylalanine, homophenylalanine, 2-methyl-phenylalanine, 3-methyl-phenylalanine, 4-methyl-phenylalanine, 2,4-dimethyl-phenylalanine, 2-nitro-phenylalanine, 3-nitro-phenylalanine, 4-nitro-phenylalanine, 2,4-dinitro-phenylalanine, 1,2,3,4-Tetrahydroisoquinoline-3-carboxylic acid, 1,2,3,4-tetrahydronorharman-3-carboxylic acid, 1-naphthylalanine, 2-naphthylalanine, pentafluorophenylalanine, 2,4-dichloro-phenylalanine, 3,4-dichloro-phenylalanine, 3,4-difluoro-phenylalanine, 3,5-difluoro-phenylalanine, 2,4,5-trifluoro-phenylalanine, 2-trifluoromethyl-phenylalanine, 3-trifluoromethyl-phenylalanine, 4-trifluoromethyl-phenylalanine, 2-cyano-phenyalanine, 3-cyano-phenyalanine, 4-cyano-phenyalanine, 2-iodo-phenyalanine, 3-iodo-phenyalanine, 4-iodo-phenyalanine, 4-methoxyphenylalanine, 2-aminomethyl-phenylalanine, 3-aminomethyl-phenylalanine, 4-aminomethyl-phenylalanine, 2-carbamoyl-phenylalanine, 3-carbamoyl-phenylalanine, 4-carbamoyl-phenylalanine, m-tyrosine, 4-amino-phenylalanine, styrylalanine, 2-amino-5-phenyl-pentanoic acid, 9-anthrylalanine, 4-tert-butyl-phenylalanine, 3,3-diphenylalanine, 4,4′-diphenylalanine, benzoylphenylalanine, α-methyl-phenylalanine, α-methyl-4-fluoro-phenylalanine, 4-thiazolylalanine, 3-benzothienylalanine, 2-thienylalanine, 2-(5-bromothienyl)-alanine, 3-thienylalanine, 2-furylalanine, 2-pyridylalanine, 3-pyridylalanine, 4-pyridylalanine, 2,3-diaminopropionic acid, 2,4-diaminobutyric acid, allylglycine, 2-amino-4-bromo-4-pentenoic acid, propargylglycine, 4-aminocyclopent-2-enecarboxylic acid, 3-aminocyclopentanecarboxylic acid, 7-amino-heptanoic acid, dipropylglycine, pipecolic acid, azetidine-3-carboxylic acid, cyclopropylglycine, cyclopropylalanine, 2-methoxy-phenylglycine, 2-thienylglycine, 3-thienylglycine, α-benzyl-proline, α-(2-fluoro-benzyl)-proline, α-(3-fluoro-benzyl)-proline, α-(4-fluoro-benzyl)-proline, α-(2-chloro-benzyl)-proline, α-(3-chloro-benzyl)-proline, α-(4-chloro-benzyl)-proline, α-(2-bromo-benzyl)-proline, α-(3-bromo-benzyl)-proline, α-(4-bromo-benzyl)-proline, α-phenethyl-proline, α-(2-methyl-benzyl)-proline, α-(3-methyl-benzyl)-proline, α-(4-methyl-benzyl)-proline, α-(2-nitro-benzyl)-proline, α-(3-nitro-benzyl)-proline, α-(4-nitro-benzyl)-proline, α-(1-naphthalenylmethyl)-proline, α-(2-naphthalenylmethyl)-proline, α-(2,4-dichloro-benzyl)-proline, α-(3,4-dichloro-benzyl)-proline, α-(3,4-difluoro-benzyl)-proline, α-(2-trifluoromethyl-benzyl)-proline, α-(3-trifluoromethyl-benzyl)-proline, α-(4-trifluoromethyl-benzyl)-proline, α-(2-cyano-benzyl)-proline, α-(3-cyano-benzyl)-proline, α-(4-cyano-benzyl)-proline, α-(2-iodo-benzyl)-proline, α-(3-iodo-benzyl)-proline, α-(4-iodo-benzyl)-proline, α-(3-phenyl-allyl)-proline, α-*-(3-phenyl-propyl)-proline, α-(4-tert-butyl-benzyl)-proline, α-benzhydryl-proline, α-(4-biphenylmethyl)-proline, α-(4-thiazolylmethyl)-proline, α-(3-benzo[b]thiophenylmethyl)-proline, α-(2-thiophenylmethyl)-proline, α-(5-bromo-2-thiophenylmethyl)-proline, α-(3-thiophenylmethyl)-proline, α-(2-furanylmethyl)-proline, α-(2-pyridinylmethyl)-proline, α-(3-pyridinylmethyl)-proline, α-(4-pyridinylmethyl)-proline, α-allyl-proline, α-propynyl-proline, γ-benzyl-proline, γ-(2-fluoro-benzyl)-proline, γ-(3-fluoro-benzyl)-proline, γ-(4-fluoro-benzyl)-proline, γ-(2-chloro-benzyl)-proline, γ-(3-chloro-benzyl)-proline, γ-(4-chloro-benzyl)-proline, γ-(2-bromo-benzyl)-proline, γ-(3-bromo-benzyl)-proline, γ-(4-bromo-benzyl)-proline, γ-(2-methyl-benzyl)-proline, γ-(3-methyl-benzyl)-proline, γ-(4-methyl-benzyl)-proline, γ-(2-nitro-benzyl)-proline, γ-(3-nitro-benzyl)-proline, γ-(4-nitro-benzyl)-proline, γ-(1-naphthalenylmethyl)-proline, γ-(2-naphthalenylmethyl)-proline, γ-(2,4-dichloro-benzyl)-proline, γ-(3,4-dichloro-benzyl)-proline, γ-(3,4-difluoro-benzyl)-proline, γ-(2-trifluoromethyl-benzyl)-proline, γ-(3-trifluoromethyl-benzyl)-proline, γ-(4-trifluoromethyl-benzyl)-proline, γ-(2-cyano-benzyl)-proline, γ-(3-cyano-benzyl)-proline, γ-(4-cyano-benzyl)-proline, γ-(2-iodo-benzyl)-proline, γ-(3-iodo-benzyl)-proline, γ-(4-iodo-benzyl)-proline, γ-(3-phenyl-allyl-benzyl)-proline, γ-(3-phenyl-propyl-benzyl)-proline, γ-(4-tert-butyl-benzyl)-proline, γ-benzhydryl-proline, γ-(4-biphenylmethyl)-proline, γ-(4-thiazolylmethyl)-proline, γ-(3-benzothioienylmethyl)-proline, γ-(2-thienylmethyl)-proline, γ-(3-thienylmethyl)-proline, γ-(2-furanylmethyl)-proline, γ-(2-pyridinylmethyl)-proline, γ-(3-pyridinylmethyl)-proline, γ-(4-pyridinylmethyl)-proline, γ-allyl-proline, γ-propynyl-proline, trans-4-phenyl-pyrrolidine-3-carboxylic acid, trans-4-(2-fluoro-phenyl)-pyrrolidine-3-carboxylic acid, trans-4-(3-fluoro-phenyl)-pyrrolidine-3-carboxylic acid, trans-4-(4-fluoro-phenyl)-pyrrolidine-3-carboxylic acid, trans-4-(2-chloro-phenyl)-pyrrolidine-3-carboxylic acid, trans-4-(3-chloro-phenyl)-pyrrolidine-3-carboxylic acid, trans-4-(4-chloro-phenyl)-pyrrolidine-3-carboxylic acid, trans-4-(2-bromo-phenyl)-pyrrolidine-3-carboxylic acid, trans-4-(3-bromo-phenyl)-pyrrolidine-3-carboxylic acid, trans-4-(4-bromo-phenyl)-pyrrolidine-3-carboxylic acid, trans-4-(2-methyl-phenyl)-pyrrolidine-3-carboxylic acid, trans-4-(3-methyl-phenyl)-pyrrolidine-3-carboxylic acid, trans-4-(4-methyl-phenyl)-pyrrolidine-3-carboxylic acid, trans-4-(2-nitro-phenyl)-pyrrolidine-3-carboxylic acid, trans-4-(3-nitro-phenyl)-pyrrolidine-3-carboxylic acid, trans-4-(4-nitro-phenyl)-pyrrolidine-3-carboxylic acid, trans-4-(1-naphthyl)-pyrrolidine-3-carboxylic acid, trans-4-(2-naphthyl)-pyrrolidine-3-carboxylic acid, trans-4-(2,5-dichloro-phenyl)-pyrrolidine-3-carboxylic acid, trans-4-(2,3-dichloro-phenyl)-pyrrolidine-3-carboxylic acid, trans-4-(2-trifluoromethyl-phenyl)-pyrrolidine-3-carboxylic acid, trans-4-(3-trifluoromethyl-phenyl)-pyrrolidine-3-carboxylic acid, trans-4-(4-trifluoromethyl-phenyl)-pyrrolidine-3-carboxylic acid, trans-4-(2-cyano-phenyl)-pyrrolidine-3-carboxylic acid, trans-4-(3-cyano-phenyl)-pyrrolidine-3-carboxylic acid, trans-4-(4-cyano-phenyl)-pyrrolidine-3-carboxylic acid, trans-4-(2-methoxy-phenyl)-pyrrolidine-3-carboxylic acid, trans-4-(3-methoxy-phenyl)-pyrrolidine-3-carboxylic acid, trans-4-(4-methoxy-phenyl)-pyrrolidine-3-carboxylic acid, trans-4-(2-hydroxy-phenyl)-pyrrolidine-3-carboxylic acid, trans-4-(3-hydroxy-phenyl)-pyrrolidine-3-carboxylic acid, trans-4-(4-hydroxy-phenyl)-pyrrolidine-3-carboxylic acid, trans-4-(2,3-dimethoxy-phenyl)-pyrrolidine-3-carboxylic acid, trans-4-(3,4-dimethoxy-phenyl)-pyrrolidine-3-carboxylic acid, trans-4-(3,5-dimethoxy-phenyl)-pyrrolidine-3-carboxylic acid, trans-4-(2-pyridinyl)-pyrrolidine-3-carboxylic acid, trans-4-(3-pyridinyl)-pyrrolidine-3-carboxylic acid, trans-4-(6-methoxy-3-pyridinyl)-pyrrolidine-3-carboxylic acid, trans-4-(4-pyridinyl)-pyrrolidine-3-carboxylic acid, trans-4-(2-thienyl)-pyrrolidine-3-carboxylic acid, trans-4-(3-thienyl)-pyrrolidine-3-carboxylic acid, trans-4-(2-furanyl)-pyrrolidine-3-carboxylic acid, trans-4-isopropyl-pyrrolidine-3-carboxylic acid, 4-phosphonomethyl-phenylalanine, benzyl-phosphothreonine, (1′-amino-2-phenyl-ethyl)oxirane, (1′-amino-2-cyclohexyl-ethyl)oxirane, (1′-amino-2-[3-bromo-phenyl]ethyl)oxirane, (1′-amino-2-[4-(benzyloxy)phenyl]ethyl)oxirane, (1′-amino-2-[3,5-difluoro-phenyl]ethyl)oxirane, (1′-amino-2-[4-carbamoyl-phenyl]ethyl)oxirane, (1′-amino-2-[benzyloxy-ethyl])oxirane, (1′-amino-2-[4-nitro-phenyl]ethyl)oxirane, (1′-amino-3-phenyl-propyl)oxirane, (1′-amino-3-phenyl-propyl)oxirane, and/or salts and/or protecting group variants thereof.

The term “peptide derivative,” as used herein, refers to a peptide comprising additional chemical or biochemical moieties not normally a part of a naturally occurring peptide. Peptide derivatives include peptides in which the amino-terminus and/or the carboxy-terminus and/or one or more amino acid side chain has been derivatised with a suitable chemical substituent group, as well as cyclic peptides, dual peptides, multimers of the peptides, peptides fused to other proteins or carriers, glycosylated peptides, phosphorylated peptides, peptides conjugated to lipophilic moieties (for example, caproyl, lauryl, stearoyl moieties) and peptides conjugated to an antibody or other biological ligand. Examples of chemical substituent groups that may be used to derivatise a peptide include, but are not limited to, alkyl, cycloalkyl and aryl groups; acyl groups, including alkanoyl and aroyl groups; esters; amides; halogens; hydroxyls; carbamyls, and the like. The substituent group may also be a blocking group such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—. Other derivatives include C-terminal hydroxymethyl derivatives, O-modified derivatives (for example, C-terminal hydroxymethyl benzyl ether) and N-terminally modified derivatives including substituted amides such as alkylamides and hydrazides. The substituent group may be a “protecting group” as detailed herein.

The term “peptidomimetic,” as used herein, refers to a compound that is structurally similar to a peptide and contains chemical moieties that mimic the function of the peptide. For example, if a peptide contains two charged chemical moieties having functional activity, a mimetic places two charged chemical moieties in a spatial orientation and constrained structure so that the charged chemical function is maintained in three-dimensional space. The term peptidomimetic thus is intended to include isosteres. The term “isostere,” as used herein, refers to a chemical structure that can be substituted for a peptide because the steric conformation of the chemical structure is similar, for example, the structure fits a binding site specific for the peptide. Examples of peptidomimetics include peptides comprising one or more backbone modifications (i.e., amide bond mimetics), which are well known in the art. Examples of amide bond mimetics include, but are not limited to, —CH₂NH—, —CH₂S—, —CH₂CH₂—, —CH═CH— (cis and trans), —COCH₂—, —CH(OH)CH₂—, —CH₂SO—, —CS—NH— and —NH—OC— (i.e., a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J; Chem. Soc. Perkin Trans. 1, 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)). Other examples of peptidomimetics include peptides substituted with one or more benzodiazepine molecules (see, for example, James, G. L. et al. (1993) Science 260:1937-1942) and peptides comprising backbones cross-linked to form lactams or other cyclic structures.

The term “variant peptide,” as used herein, refers to a peptide in which one or more amino acid residue has been deleted, added or substituted in comparison to the amino acid sequence to which the peptide corresponds. Typically, when a variant contains one or more amino acid substitutions they are “conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include: alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group.

The terms “percent (%) amino acid sequence identity” or “percent amino acid sequence homology” as used herein with respect to a reference polypeptide is defined as the percentage of amino acid residues in a candidate peptide sequence that are identical with the amino acid residues in the reference polypeptide sequence after aligning the sequences and introducing gaps, if necessary, to achieve the maximum percent sequence identity, without considering any conservative substitutions as part of the sequence identity. Alignment for the purpose of determining percent amino acid sequence identity can be achieved by various techniques known in the art, for instance, using publicly available computer software such as ALIGN or Megalign (DNASTAR). Those skilled in the art can determine appropriate parameters for measuring alignment, including any algorithms needed to achieve maximal alignment over the full length of the peptide sequence being used in the comparison. An analogue is said to share “substantial homology” if the amino acid sequences of said compound are at least 80%, and more preferably at least 90%, and most preferably at least 95%, the same as that of the subject sequence of comparison.

The phrase “protecting group” as used herein, means temporary substituents which protect a potentially reactive functional group from undesired chemical transformations.

The term “amino-protecting group” or “N-terminal protecting group” refers to those groups intended to protect the α-N-terminal of an amino acid or peptide or to otherwise protect the amino group of an amino acid or peptide against undesirable reactions during synthetic procedures. Commonly used N-protecting groups are disclosed in Greene, Protective Groups In Organic Synthesis, (John Wiley & Sons, New York (1981)), which is hereby incorporated by reference. Additionally, protecting groups can be used as pro-drugs which are readily cleaved in vivo, for example, by enzymatic hydrolysis, to release the biologically active parent. α-N-Protecting groups comprise lower alkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyoxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; arylalkyl groups such as benzyl, triphenylmethyl, benzyloxymethyl, 9-fluorenylmethyloxycarbonyl (Fmoc) and the like and silyl groups such as trimethylsilyl and the like. Still other examples include theyl, succinyl, methoxysuccinyl, subery, adipyl, azelayl, dansyl, benzyloxycarbonyl, methoxyazelaly, methoxyadipyl, methoxysuberyl, and 2,4-dinitrophenyl.

The term “carboxy protecting group” or “C-terminal protecting group” refers to a carboxylic acid protecting ester or amide group employed to block or protect the carboxylic acid functionality while the reactions involving other functional sites of the compound are performed. Carboxy protecting groups are disclosed in Greene, Protective Groups in Organic Synthesis pp. 152-186 (1981), which is hereby incorporated by reference. Additionally, a carboxy protecting group can be used as a pro-drug whereby the carboxy protecting group can be readily cleaved in vivo, for example by enzymatic hydrolysis, to release the biologically active parent. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated herein by reference. Representative carboxy protecting groups are C₁-C₈ loweralkyl (e.g., methyl, ethyl or t-butyl and the like); arylalkyl such as phenethyl or benzyl and substituted derivatives thereof such as alkoxybenzyl or nitrobenzyl groups and the like; arylalkenyl such as phenylethenyl and the like; aryl and substituted derivatives thereof such as 5-indanyl and the like; dialkylaminoalkyl such as dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl and the like; aroyloxyalkyl such as benzoyloxymethyl, benzoyloxyethyl and the like; arylalkylcarbonyloxyalkyl such as benzylcarbonyloxymethyl, 2-benzylcarbonyloxyethyl and the like; alkoxycarbonylalkyl or cycloalkyloxycarbonylalkyl such as methoxycarbonylmethyl, cyclohexyloxycarbonylmethyl, 1-methoxycarbonyl-1-ethyl and the like; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, 1-cyclohexyloxycarbonyloxy-1-ethyl and the like; aryloxycarbonyloxyalkyl such as 2-(phenoxycarbonyloxy)ethyl, 2-(5-indanyloxycarbonyloxy)ethyl and the like; alkoxyalkylcarbonyloxyalkyl such as 2-(1-methoxy-2-methylpropan-2-oyloxy)ethyl and like; arylalkyloxycarbonyloxyalkyl such as 2-(benzyloxycarbonyloxy)ethyl and the like; arylalkenyloxycarbonyloxyalkyl such as 2-(3-phenylpropen-2-yloxycarbonyloxy)ethyl and the like; alkoxycarbonylaminoalkyl such as t-butyloxycarbonylaminomethyl and the like; alkylaminocarbonylaminoalkyl such as methylaminocarbonylaminomethyl and the like; alkanoylaminoalkyl such as acetylaminomethyl and the like; heterocycliccarbonyloxyalkyl such as 4-methylpiperazinylcarbonyloxymethyl and the like; dialkylaminocarbonylalkyl such as dimethylaminocarbonylmethyl, diethylaminocarbonylmethyl and the like; (5-(loweralkyl)-2-oxo-1,3-dioxolen-4-yl)alkyl such as (5-t-butyl-2-oxo-1,3-dioxolen-4-yl)methyl and the like; and (5-phenyl-2-oxo-1,3-dioxolen-4-yl)alkyl such as (5-phenyl-2-oxo-1,3-dioxolen-4-yl)methyl and the like. Representative amide carboxy protecting groups are aminocarbonyl and loweralkylaminocarbonyl groups. For example, aspartic acid may be protected at the α-C-terminal by an acid labile group (e.g., t-butyl) and protected at the β-C-terminal by a hydrogenation labile group (e.g., benzyl) then deprotected selectively during synthesis. As mentioned above, the protected carboxy group may also be a loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The terms “Lewis base” and “Lewis basic” are recognized in the art, and refer to a chemical moiety capable of donating a pair of electrons under certain reaction conditions. Examples of Lewis basic moieties include uncharged compounds such as alcohols, thiols, olefins, and amines, and charged moieties such as alkoxides, thiolates, carbanions, and a variety of other organic anions.

The terms “Lewis acid” and “Lewis acidic” are art-recognized and refer to chemical moieties which can accept a pair of electrons from a Lewis base.

The term “regioisomers” refers to compounds which have the same molecular formula but differ in the connectivity of the atoms. Accordingly, a “regioselective process” is one which favors the production of a particular regioisomer over others, e.g., the reaction produces a statistically significant preponderance of a certain regioisomer.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluoromethyl), cyano, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho (o-), meta (m-) and para (p-) are art-recognized and apply to 1,2-, 1,3- and 1,4-disubstituted benzenes, respectively. For example, the names 1,2-dimethylbenzene, ortho-dimethylbenzene and o-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, fluoroalkyl (such as trifluoromethyl), cyano, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, fluoroalkyl (such as trifluoromethyl), cyano, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The term “acyl” is art-recognized and refers to any group or radical of the form RCO— where R is any organic group. Representative acyl group include acetyl, benzoyl, and malonyl.

The term “acyloxy” is art-recognized and refers to a moiety that can be represented by the general formula:

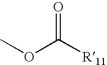

wherein R′₁₁ represents a hydrogen, an alkyl, an aryl, an alkenyl, an alkynyl or —(CH₂)ₘ—R₈, where m is 1-30 and R₈ represents a group permitted by the rules of valence.

The terms “amine” and “amino” are art-recognized and include both unsubstituted and substituted amines, e.g., a moiety that may be represented by the general formulas:

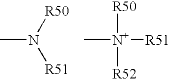

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

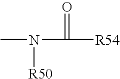

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

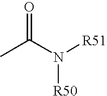

wherein R50 and R51 are as defined above. Certain embodiments of the amide in the present invention will not include amides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethylthio, and the like.

The term “carbonyl” is art recognized and includes such moieties as may be represented by the general formulas:

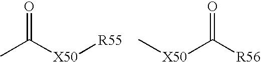

wherein X50 is a bond or represents an oxygen or a sulfur, and R55 represents a hydrogen, an alkyl, an alkenyl, —(CH₂)—, —R61 or a pharmaceutically acceptable salt, R56 represents a hydrogen, an alkyl, an alkenyl or —(CH₂)ₘ—R61, where m and R61 are defined above. Where X50 is an oxygen and R55 is not hydrogen, the formula represents an “ester”. Where X50 is an oxygen, and R55 is as first defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “oxime” and “oxime ether” are art-recognized and refer to moieties that may be represented by the general formula:

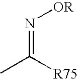

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and includes a moiety that may be represented by the general formula:

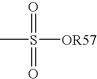

in which R57 is an electron pair, hydrogen, alkyl, cycloalkyl, or aryl.

The term “sulfate” is art recognized and includes a moiety that may be represented by the general formula:

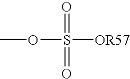

in which R57 is as defined above.

The term “sulfonamido” is art recognized and includes a moiety that may be represented by the general formula:

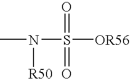

in which R50 and R56 are as defined above.

The term “sulfamoyl” is art-recognized and includes a moiety that may be represented by the general formula:

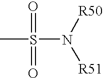

in which R50 and R51 are as defined above.

The term “sulfonyl” is art recognized and includes a moiety that may be represented by the general formula:

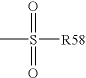

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

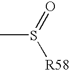

in which R58 is defined above.

The term “phosphoryl” is art-recognized and may in general be represented by the formula:

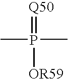

wherein Q50 represents S or O, and R59 represents hydrogen, a lower alkyl or an aryl. When used to substitute, e.g., an alkyl, the phosphoryl group of the phosphorylalkyl may be represented by the general formulas:

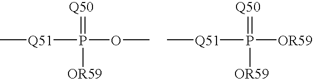

wherein Q50 and R59, each independently, are defined above, and Q51 represents O, S or N. When Q50 is S, the phosphoryl moiety is a “phosphorothioate.”

The term “phosphoramidite” is art recognized and includes moieties represented by the general formulas:

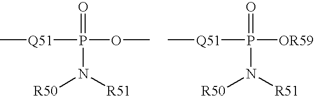

wherein Q51, R50, R51 and R59 are as defined above.

The term “phosphonamidite” is art recognized and includes moieties represented by the general formulas:

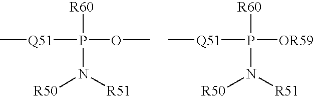

wherein Q51, R50, R51 and R59 are as defined above, and R60 represents a lower alkyl or an aryl.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms, represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds contained in compositions of the present invention may exist in particular geometric or stereoisomeric forms. In addition, compounds of the present invention may also be optically active. The present invention contemplates all such compounds, including cis- and trans-isomers, (R)- and (S)-enantiomers, diastereoisomers, (D)-isomers, (L)-isomers, the racemic mixtures thereof; and other mixtures thereof, as falling within the scope of the invention. Additional asymmetric carbon atoms may be present in a substituent such as an alkyl group. All such isomers, as well as mixtures thereof; are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g., alkyl, m, n, etc., when it occurs more than once in any structure, is intended to be independent of its definition elsewhere in the same structure unless otherwise indicated expressly or by the context.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

### Compounds of the Invention

The present invention relates, in part, to a compound represented by formula 1:

A-(B)ₙ—C 1

or a pharmaceutically acceptable salt or composition thereof;

wherein
A represents an oligopeptide sequence that is a portion or all of a substrate for an activating protease, wherein said oligopeptide sequence comprises a bond cleavable by said activating protease;
B is independently for each occurrence a D-isomer or L-isomer of a naturally occurring alpha-amino acid;
C represents a chemotherapeutic agent; and
n is an integer in the range of zero to about five, and to compositions thereof.

Of importance to the compounds of the present invention is the binding of a chemotherapeutic agent with a substrate of an activating protease. The chemotherapeutic agent may be bound to the substrate of an activating protease either directly or through an amino acid linker, B. The result is the local delivery of the chemotherapeutic agent to the vicinity of the activating protease. When the activating protease is one generally found in cancer cells, such as FAP, the result is the local delivery of the chemotherapeutic agent to the cancer cells. The compounds of the present invention therefore offer an improved treatment for cancer over systemic administration.

The chemotherapeutic agent may be bound to the oligopeptide sequence through commonly known organic chemistry functional groups, but given the peptide nature of the sequence, the preferred method of binding the chemotherapeutic agent to the substrate is through ester, amide, or carboxamide linkages. Useful functional groups on either the oligopeptide sequence or chemotherapeutic agent would therefore include amino, hydroxy, or carboxylic acid groups. For example, paclitaxel or docetaxel could be bound via an ester linkage between one of its hydroxy groups and the C-terminus of the oligopeptide sequence. Other details regarding the various components of the compounds of the present invention follow.

### Proteases

Proteases (also known as proteinases, peptidases or proteolytic enzymes) are enzymes that break peptide bonds between amino acids of proteins, a process called proteolytic cleavage. Generally, the present invention can be adapted to wide range of proteases depending on the application. Oligopeptide sequences that act as substrates for the various proteases are generally known to one of ordinary skill in the art.

There are currently six classes of proteases: serine proteases, threonine proteases, cysteine proteases, aspartic acid proteases, metalloproteases, and glutamic acid proteases. The threonine and glutamic proteases were not described until 1995 and 2004, respectively. The mechanism used to cleave a peptide bond involves making an amino acid residue (serine, cysteine and threonine peptidases) or a water molecule (aspartic, metallo and glutamic peptidases) nucleophilic so that it can attack the peptide carbonyl group. One way to make a nucleophile is by a catalytic triad, where a histidine residue is used to activate serine, cysteine or threonine as a nucleophile.

Non-limiting examples of serine proteases include FAP, dipeptidyl-peptidase IV, acylaminoacyl-peptidase, lysosomal Pro-Xaa carboxypeptidase, nucleoporin, and lactoferrin. Non-limiting examples of threonine proteases include glycosylasparaginase precursor. Non-limiting examples of cysteine proteases include calpain-2, poliovirus-type picornain 3C, poliovirus-type picornain 2A, parechovirus picornain 3C, adenain, ubiquitinyl hydrolase-L1, ubiquitin-specific peptidase 14, amidophosphoribosyltransferase precursor, autophagin-1, Cezanne deubiquitinating peptidase, otubain-1, CylD protein, and UL36 deubiquitinylating peptidase. Non-limiting examples of aspartic acid proteases include pepsin A, HIV-1 retropepsin, spumapepsin, presenilin 1, and impas 1 peptidase. Non-limiting examples of metalloproteases include aminopeptidase N, angiotensin-converting enzyme peptidase unit 1, matrix metallopeptidase-1, neprilysin, carboxypeptidase A1, eupitrilysin, membrane dipeptidase, pappalysin-1, S2P peptidase, and AMSH deubiquitinating peptidase. Non-limiting examples of glutamic acid proteases include scytalidoglutamic peptidase.

Proteases, such as FAP, that play a role in either the proliferation of cancer cells or in their resistance to treatment or that are otherwise associated with cancer cells are preferred. More examples of such proteases may be found in Barrett A. J., Rawlings N. D., Woessner J. F. The Handbook of Proteolytic Enzymes, 2nd ed. Academic Press, 2003, incorporated herein by reference in its entirety.

### FAP Substrate Specificity

The terminology used herein for designating substrate sets based on enzyme specificity pockets is in accordance with that used in the art, whereby P₁-P′₁ represents the scissile bond (e.g., site of FAP cleavage), Pₙ₊₁ represents residues N-terminal to the scissile bond, and P′ₙ₊₁ represents residues C-terminal to the scissile bond:

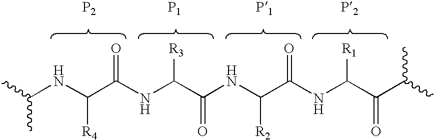

Edosada et al. contend that a small amino acid, such as D-Ala, can substitute for Gly, and that with respect to FAP activity Ser is like Ala in possessing a small side chain (vide supra). Indeed, preliminary analysis of available models of the enzyme-inhibitor complex would seem to preclude larger D-amino acids due to steric clashes, as the methyl group of D-Ala is demonstrated to be protruding into the enzyme. However, based on a rigorous structural analysis, Aertgeerts et al. suggest that a FAP active site should “accommodate most amino acids” (vide supra). In an effort to resolve these conflicting reports, studies were undertaken to explore further FAP substrate specificity with an eye toward developing a range of therapeutic agents that would exploit said characteristics.

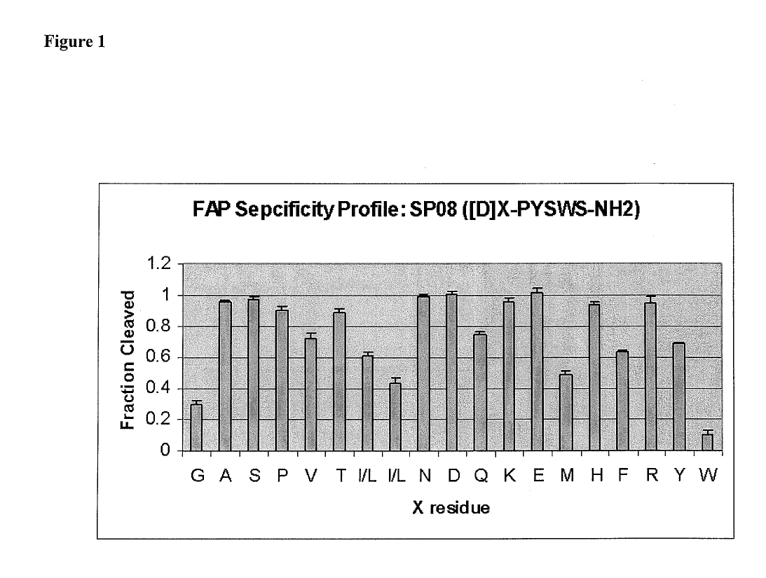

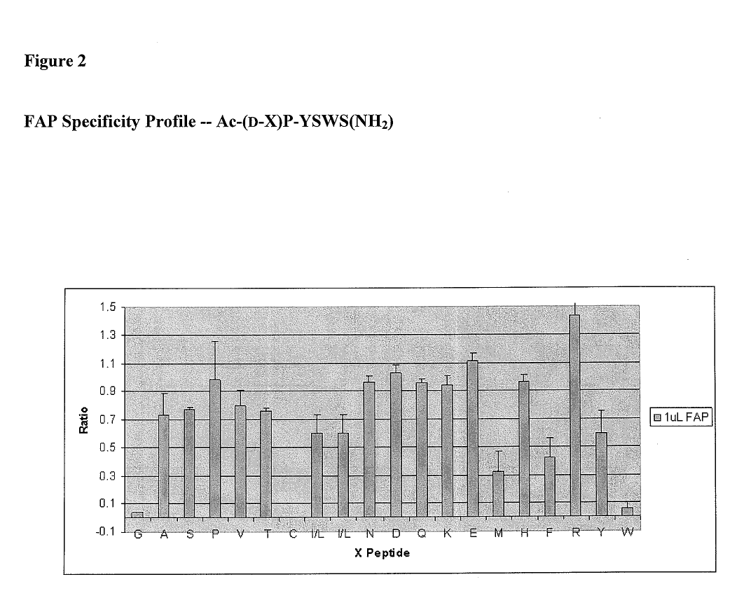

FIGS. 1 and 2 show the results for FAP cleavage of P₂-P′₄ hexapeptide derivatives XP-YSWS in libraries of substrates, wherein the amino acid residues being screened for efficacy in a P₂ substitution (represented by X) are all in the D-configuration. For the library represented in FIG. 1, the N-terminus of said hexapeptide is unprotected; for the library represented in FIG. 2, the N-terminus of said hexapeptide is acetylated. In both libraries, all of the hexapeptides have been modified at the C-terminal carboxylate to include a geminal-amino functionality. In both libraries, the P₁ residue is Pro.

The test substrates were incubated with FAP at 37° C. for 24 hours. Substrate hydrolysis occurs at the scissile bond between P₁ (Pro) and P′₁ (Tyr). The reaction was quenched with 1.2 N HCl, and the samples were analyzed by LC/MS to determine the amount of intact peptide remaining compared to the library. The fraction cleaved is depicted for each D-amino acid residue X. Taken together, the data for all analyses indicate that D-tryptophan is the best P₂ residue, regardless of the absence or presence of N-terminal protection.

### Chemotherapeutic Agents

The present invention relates to chemotherapeutic agents bound to a peptidyl substrate for an activating protease. The chemotherapeutic agent is generally bound to the C terminus of the peptide sequence and thus forms a bond with the CO group at that terminus, or, in the embodiments where the C terminus has been modified, to some other electrophilic group. Any of the chemotherapeutic agents known in the art that contain a nucleophilic group capable of forming a bond with an electrophilic group can potentially be used. Examples of nucleophilic groups include groups that contain an N, O, or S atom such as, for example, an amino, hydroxy, or thiol group.

The following discussion is of some of the more common chemotherapeutic agents used for the treatment of cancer. The examples listed are not meant to be limiting in any way nor do they fully represent the full range of chemotherapeutic agents that potentially may be used in the present invention.

### Alkylating Agents

Alkylating agents are so named because of their ability to add alkyl groups to many electronegative groups under conditions present in cells. They stop tumor growth by cross-linking guanine nucleobases in DNA double-helix strands—directly attacking DNA. This makes the strands unable to uncoil and separate. As this is necessary in DNA replication, the cells can no longer divide. These drugs act nonspecifically. Some of them require conversion into active substances in vivo (e.g., cyclophosphamide).

Cyclophosphamide is one of the most potent immunosuppressive substances. In small dosages, it is very efficient in the therapy of systemic lupus erythematosus, autoimmune hemolytic anemias, Wegener's granulomatosis and other autoimmune diseases. High dosages cause pancytopenia and hemorrhagic cystitis.

Dialkylating agents can react with two different 7-N-guanine residues and if these are in different strands of DNA the result is cross-linkage of the DNA strands, which prevents uncoiling of the DNA double helix. If the two guanine residues are in the same strand the result is called limpet attachment of the drug molecule to the DNA. Monoalkylating agents can react only with one 7-N of guanine. Limpet attachment and monoalkylation do not prevent the separation of the two DNA stands of the double helix but do prevent vital DNA processing enzymes from accessing the DNA. The final result is inhibition of cell growth or stimulation of apoptosis, cell suicide. Since cancer cells generally divide more rapidly than do healthy cells they are more sensitive to DNA damage, and alkylating agents are used clinically to treat a variety of tumors.

Non-limiting examples of alkylating agents include cyclophosphamide, chlorambucil, estramustine, melphalan, mechlorethamine, ifosfamide, busulfn, treosulfan, thioTEPA, lomustine, carmustine, streptozocin, temozolomide, dacarbazine, cisplatin, carboplatin, oxaliplatin, procarbazine, trofosfamide, and uramustine.

### Antimetabolites

An antimetabolite is a chemical with a similar structure to a substance (a metabolite) required for normal biochemical reactions, yet different enough to interfere with the normal functions of cells, including cell division. Antimetabolites can be used in cancer treatment, as they interfere with DNA production and therefore cell division and the growth of tumors.

Anti-metabolites masquerade as purine (azathioprine, mercaptopurine) or pyrimidine—which become the building blocks of DNA. They prevent these substances becoming incorporated in to DNA during the “S” phase (of the cell cycle), stopping normal development and division. They also affect RNA synthesis. Due to their efficiency, these drugs are the most widely used cytostatics.

Antimetabolites may also be antibiotics, such as sulfanilamide drugs, which inhibit dihydrofolate synthesis in bacteria by competing with para-aminobenzoic acid. (Silverman, 2004).

Non-limiting examples of antimetabolites include methotrexate, pemetrexed, fludarabine cytarabine, 5-fluorouracil, floxuridine, gemcitabine, trimetrexate, mycophenolic acid, tiazofurin, ribavirin, EICAR, doxifluridine, ratitrexed, cytosine arabinoside, mercaptopurine, thioquanine, and capecitabine.

### Plant Alkaloids

These alkaloids are derived from plants and block cell division by preventing microtubule function. Microtubules are vital for cell division and without them it can not occur. The main examples are vinca alkaloids and taxanes.

Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea).

Podophyllotoxin is a plant-derived compound used to produce two other cytostatic drugs, etoposide and teniposide. They prevent the cell from entering the G1 phase (the start of DNA replication) and the replication of DNA (the S phase). The substance has been primarily obtained from the American Mayapple (Podophyllum peltatum). Recently it has been discovered that a rare Himalayan Mayapple (Podophyllum hexandrum) contains it in a much greater quantity, but as the plant is endangered, its supply is limited. Studies have been conducted to isolate the genes involved in the substance's production, so that it could be obtained recombinantly.

Taxanes are derived from the Yew Tree. Paclitaxel is derived from the bark of the European Yew Tree while docetaxel is derived from the pine needle of the Pacific Yew Tree. Taxanes enhance stability of microtubules, preventing the separation of chromosomes during anaphase.

Non-limiting examples of plant alkaloids include vinblastine, vincristine, vinorelbine, vindesine, etoposide, teniposide, paclitaxel, and docetaxel.

### Topoisomerase Inhibitors

Topoisomerases are essential enzymes that maintain the topology of DNA. Inhibition of type I or type II topoisomerases interferes with both transcription and replication of DNA by upsetting proper DNA supercoiling.

Non-limiting examples of topoisomerase inhibitors include amsacrin, etoposide, teniposide, 9-aminocamptothecin, crisnatol, topotecan and irinotecan.

### Antitumor Antibiotics

Antitumor antibiotics are drugs that inhibit and combat the development of tumors. Antitumor antibiotics work by: 1) inhibiting topoisomerase II which stops DNA being unwound, which is required for both DNA replication and RNA/protein synthesis, and 2) generating free radicals. They are products of various strains of the soil fungus Streptomyces.

Non-limiting examples of antitumor antibiotics include actinomycin, dactinomycin, doxorubicin, pirarubicin, zorubicin, daunorubicin, epirubicin, idarubicin, mitoxantrone, bleomycin A2, bleomycin. B2, peplomycin, mitomycin C, plicamycin, deferoxamine, and hydroxyurea.

### Hormonal and Receptor Antagonists

Non-limiting examples of hormones and receptor antagonists that can be used as chemotherapeutics include tamoxifen, raloxifene, megestrol, goserclin, leuprolide acetate, flutamide, and bicalutamide.

### Retinoids and Deltoids

Non-limiting examples of retinoids and deltoids that can be used as chemotherapeutics include EB 1089, CB 1093, KH 1060, vertoporfin, phthalocyanine, photosensitizer Pc4, demethoxy-hypocrellin A, interferon α, interferon γ, and tumor necrosis factor.

### Others

Non-limiting examples of other chemotherapeutics that may be used in the present invention include isoprenylation inhibitors such as lovastatin; dopaminergic neurotoxins such as 1-methyl-4-phenylpyridinium ion; cell cycle inhibitors such as staurosporine; MDR inhibitors such as verapamil; Ca²⁺ ATPase inhibitors such as thapsigargin; antibodies such as avastin, erbitux, and rituxan, and other agents such as prednisilone, imatinib, thalidomide, lenalidomide, bortezomib, gemcitabine, erlotinib, gefitinib, sorafenib, and sutinib.

Effective cancer treatment often involves administration of more than one type of chemotherapeutic agent. For example, one treatment for breast cancer includes the administration of cyclophosphamide, methotrexate, and 5-fluorouracil (CMF) intravenously. It is envisioned by the inventors that the present invention may be adapted to such multi-type treatments.

In another embodiment, the chemotherapeutic agent is an inhibitor of the activating protease. In this embodiment, the activating protease releases its own inhibitor. The prodrugs comprising this type of chemotherapeutic agent are referred to as a “smart pro-drug.”

### Smart Pro-Drugs

Aspects of the present invention may also exploit the concepts of smart pro-drug technology. For example, certain embodiments of the present invention relate to an approach to drug delivery centered on therapeutic agents of the general formula:

S-Xaa-Xaa-T

wherein S is an “address moiety,” such as a target or tissue-specific functional group or substructure; Xaa represents independently for each occurrence an amino acid; and Xaa-Xaa-T is designed for target recognition.

For example, in certain embodiments of the present invention, S is a substrate for cancer- or tissue-specific activator protease, such as FAP or PSA, and Xaa-Xaa-T is targeted for proteasome inhibition (e.g., enzyme inactivation). In certain embodiments of the present invention, T may be a geminal-amino, boronic acid, nitrile, aldehyde, α-keto amide, or trifluoromethylketone moiety, representing a C-terminal modification of a peptide sequence.

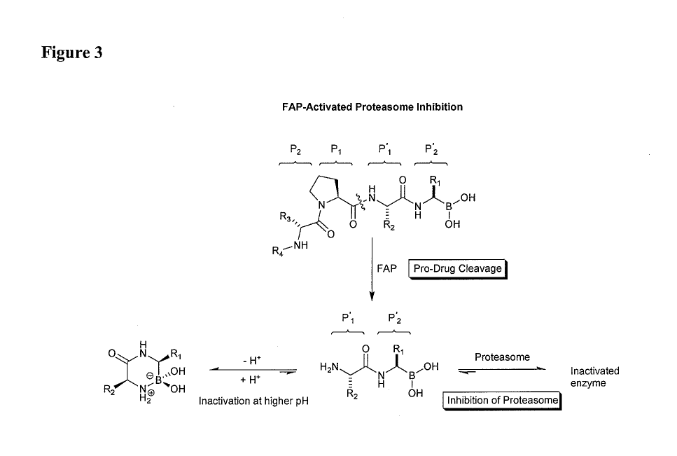

FIG. 3 depicts a scheme for FAP-activated proteasome inhibition according to the present invention. In this scheme, FAP mediates cleavage of the P₂-P′₂ tetrapeptide pro-drug at the P₁-P′₁ scissile bond after proline, effectively “releasing” a P′₁-P′₂ dipeptide boronic acid derivative, which is a proteasome inhibitor at lower pH (˜2). For example, the pro-drug Xaa-Pro-Phe-Leu-B(OH)₂, wherein Xaa may be a D- or L-amino acid (and in certain embodiments of the present invention, Xaa is D-Trp), is designed to release the pseudodipeptide NH₂-Phe-Leu-B(OH)₂ following proline-specific cleavage by FAP (Scheme 1). Said pseudodipeptide is tailored to inhibit proteasome targets.

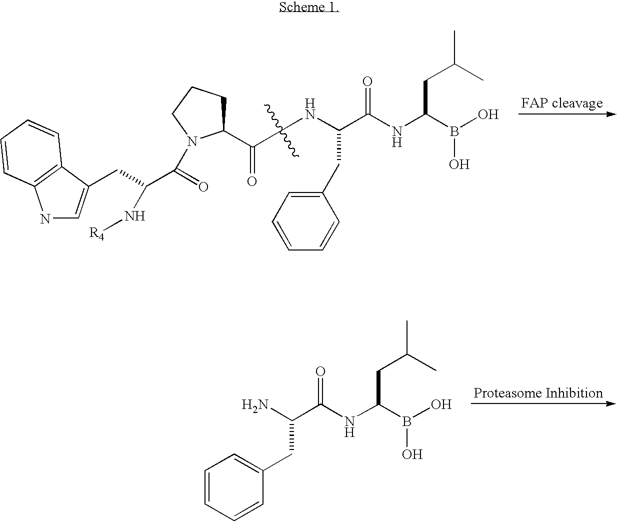

It is understood that dipeptidyl boronic acids may exist in either cyclical or linear monomeric form depending on pH, or as a trimeric boroxine, a cyclic anhydride form. Indeed, the dipeptidyl boronic acids are known to be inactivated at higher pH (˜7.6), as a consequence of monomeric cyclization (FIG. 3).

It is envisaged that said P′₁-P′₂ pseudodipeptide boronic acid derivatives of the present invention, which are released by the pro-drug parent, could be inhibitors of the chymotrypsin-like activity of the 26S proteasome in mammalian cells. The 26S proteasome is a large protein complex that degrades ubiquitinated proteins. The ubiquitin-proteasome pathway plays an essential role in regulating the intracellular concentration of specific proteins, thereby maintaining homeostasis within cells. Inhibition of the 26S proteasome prevents this targeted proteolysis, which can affect multiple signaling cascades within the cell. This disruption of normal homeostatic mechanisms can lead to cell death. For example, these therapies could cause a delay in tumor growth in vivo, including multiple myeloma. It may also be the case that deboronated species (e.g., metabolites) are inactive as inhibitors (see below).

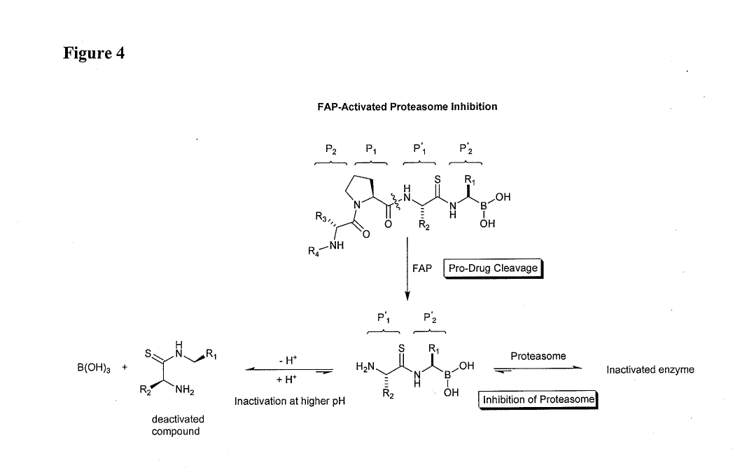

### Ultra Smart Pro-Drugs

Whereas with Smart Pro-Drugs deactivation is a function of equilibrium, Ultra Smart Pro-Drugs deactivate themselves over time via loss of a functional group. Because a covalent bond is broken, deactivation of an Ultra Smart Pro-Drug is more definite and permanent than with Smart Pro-Drugs. An example of an Ultra-Smart Pro-Drug is depicted in FIG. 4. Comparing FIG. 4 to FIG. 3, one can see that the carbonyl group of P′₁ has been replaced with a C═S group. The electronic difference between sulfur and oxygen is enough to result in the release of B(OH)₃, leaving the deactivated dipeptidyl substrate. Ultra Smart Pro-Drugs are even more innocuous towards healthy cells than Smart Pro-Drugs because as the released drugs diffuse to other areas of the body, their deactivation is more permanent and less harmful to the patient.

### Termini Modifications

The use of “protecting groups” (see Definitions) as described herein may also be advantageous. Further examples of N-terminal modifications include glycation (e.g., N-glucitol), N-pyroglutamyl, N-acetyl, N-methylation (N-Me, α-Me), desamination, and substitution with imidazole-lactic acid.

Certain embodiments of the present invention provide for synthetic peptide analogues that may be optionally derivatized at the terminal residues, independently for each occurrence. One embodiment of the present invention considers peptide analogues, as described above, which are optionally derivatized, whereby the corresponding C-terminal carboxylate is replaced with a geminal-amino or boronic acid functionality, independently for each occurrence, to afford stable, biologically active FAP-activated proteasome inhibitors.

C-Terminal modification may also consider analogous structures, such as those in which the requisite carboxylate is replaced by, for example, phosphonate, fluoroalkylketone, alphaketos, N-peptiolyl-O-(acylhydroylamines), azapeptides, azetidines, fluoroolefins, dipeptide isosteres, peptidyl (alpha-aminoalkyl) phosphonate esters, aminoacyl pyrrolidine-2-nitriles, or 4-cyanothiazolidides.

In each instance above, if the C-terminal carbon bonded to said modified functionality (e.g., boronic acid) is described as being in the L-configuration, what is meant is that the absolute configuration of said carbon is like that of an L-amino acid. For example, the group —B(OH)₂ would have the same relationship to said carbon as the —COOR group of an L-amino acid would have:

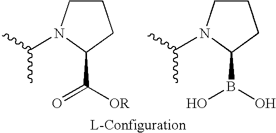

As used herein, the terminology FAP-activated proteasome inhibitor, including those of the present invention, also includes pharmaceutically acceptable salts of said compounds described, and in accordance with the detailed definitions herein. A compound of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

In the formulas representing selected specific embodiments of the present invention, the amino- and carboxy-terminal groups, although often not specifically shown, may be understood to be in the form they would assume at physiological pH values, unless otherwise specified. Thus, the N-terminal-H₂⁺ and C-terminal-O⁻ at physiological pH may be understood to be present, though not necessarily specified and shown, either in specific examples or in generic formulas.

The foregoing describes the status of the termini at neutral pH; it is understood, of course, that the acid addition salts or the basic salts of the peptides are also included within the scope of the invention. At high pH, basic salts of the C-terminus and carboxyl-containing side chains may be formed from nontoxic pharmaceutically acceptable bases, and suitable counter-ions include, for example, Na⁺, K⁺, Ca²⁺, and the like. Suitable pharmaceutically acceptable nontoxic organic cations can also be used as counter ions. In addition, as set forth herein, the peptides may be prepared as the corresponding amides. Suitable acid addition salts with respect to the N-terminus or amino group-containing side chains include the salts formed from inorganic acids such as hydrochloric, sulfuric, or phosphoric acid and those formed from organic acids such as acetic, citric, or other pharmaceutically acceptable nontoxic acids.

### Methods of the Invention

As alluded to above, it is envisaged that the FAP-activated proteasome inhibitors of the present invention should be useful as therapeutic agents for the treatment of conditions that are susceptible to amelioration by such an agent, such as those characterized by an abnormal mammalian cell proliferation, which may be manifest as a tumor. Said FAP-activated proteasome inhibitors may also be useful for treating a condition as described above, which is further characterized by the presence of reactive stromal fibroblasts. Said FAP-activated proteasome inhibitors may be useful for treating a condition as described above, wherein the abnormal mammalian cell proliferation is in epithelial cells, such as a carcinoma, a sarcoma, or a melanoma. Said FAP-activated proteasome inhibitors may also be useful for treating a condition as described above, which is further characterized by a metastasis of epithelial origin. The FAP-activated proteasome inhibitors of the present invention may be useful for the treatment of such conditions breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma. Said FAP-activated proteasome inhibitors may be useful in each instance as described above for inhibiting angiogenesis.

### Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) nasally; (9) pulmonary; or (10) intrathecally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts,” J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled-release agents, such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable non-irritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In some cases, in order to prolong the effect of a drug, it is desirable to slow the absorption of the drug from subcutaneous or intramuscular injection. This result may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers, such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, administration by injection, infusion or inhalation; topical by lotion or ointment; and rectal by suppositories.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals, such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutic effects of the first administered one is not entirely disappeared when the subsequent is administered.

Non-limiting examples of carriers include polymers and copolymers, micelles, reverse micelles, liposomes, microspheres, emulsions, hydrogels, microparticles, nanoparticles, and solid surfaces. In one aspect, the carrier is biocompatible.

### (i) Polymers and Co-polymers

In certain embodiments, the polymers or co-polymers of the subject compositions, e.g., which include repetitive elements shown in any of the subject formulas, have molecular weights ranging from about 2000 or less to about 1,000,000 or more daltons, or alternatively about 10,000, 20,000, 30,000, 40,000, or 50,000 daltons, more particularly at least about 100,000 daltons, and even more specifically at least about 250,000 daltons or even at least 500,000 daltons. Number-average molecular weight (Mn) may also vary widely, but generally fall in the range of about 1,000 to about 200,000 daltons, or even from about 1,000 to about 100,000 daltons or even from about 1,000 to about 50,000 daltons. In one embodiment, Mn varies between about 8,000 and 45,000 daltons. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In certain embodiments, the intrinsic viscosities of the polymers generally vary from about 0.01 to about 2.0 dL/g in chloroform at 40° C., alternatively from about 0.01 to about 1.0 dL/g and, occasionally, from about 0.01 to about 0.5 dL/g.

The glass transition temperature (Tg) of the subject polymers may vary widely, and depend on a variety of factors, such as the degree of branching in the polymer components, the relative proportion of phosphorous-containing monomer used to make the polymer, and the like. When the article of the invention is a rigid solid, the Tg is often within the range of from about −10° C. to about 80° C., particularly between about 0 and 50° C. and, even more particularly between about 25° C. to about 35° C. In other embodiments, the Tg is low enough to keep the composition of the invention flowable at body temperature. Then, the glass transition temperature of the polymer used in the invention is usually about 0 to about 37° C., or alternatively from about 0 to about 25° C.

In other embodiments, the polymer composition of the invention may be a flexible or flowable material. When the polymer used is itself flowable, the polymer composition of the invention, even when viscous, need not include a biocompatible solvent to be flowable, although trace or residual amounts of biocompatible solvents may still be present.

A flexible polymer may be used in the fabrication of a solid article. Flexibility involves having the capacity to be repeatedly bent and restored to its original shape. Solid articles made from flexible polymers are adapted for placement in anatomic areas where they will encounter the motion of adjacent organs or body walls. A flexible solid article can thus be sufficiently deformed by those moving tissues that it does not cause tissue damage. Flexibility is particularly advantageous where a solid article might be dislodged from its original position and thereby encounters an unanticipated moving structure; flexibility may allow the solid article to bend out of the way of the moving structure instead of injuring it. Such a flexible article might be suitable for covering pulsatile vessels such as the carotid artery in the neck, or for covering more delicate structures in the neck like the jugular vein that may also be affected by local movements. Similarly, a flexible solid article may be used to protect nerves exposed during a neck dissection such as the spinal accessory nerve, wherein the flexibility of the solid article may permit it to bend or deform when encountering motion rather than eroding into or damaging the nerve. Use of a solid carrier according to the present invention in the aforesaid ways may allow less extensive dissections to be carried out with surgical preservation of structures important to function. Solid articles may be configured as three-dimensional structures suitable for implantation in specific anatomic areas. Solid articles may be formed as films, meshes, sheets, tubes, or any other shape appropriate to the dimensions and functional requirements of the particular anatomic area. Physical properties of polymers may be adjusted to attain a desirable degree of flexibility by modification of the chemical components and crosslinking thereof, using methods familiar to practitioners of ordinary skill in the art.

Examples of polymeric carriers include carboxylated or carboxymethylated linear poly-1-lysine (PL) or poly-D-lysine; carboxylated or carboxymethylated poly-alpha, beta-(2-aminoethyl)-D,L-aspartamide; poly-1-aspartic acid; poly-glutamic acid, copolymers of histidine with positively or negatively charged aminoacids, carboxylated polyethyleneimines, i.e., polyethylene imines reacted with derivatives of carbonic acids; natural saccharides or products chemically derived thereof, bearing carboxylic groups, which may be exemplified by: galacturonic acid, glucuronic acid, mannuronic acid, hyaluronic acid, pectic acid, neuraminic acid, alginic acid, carrageenan; oxidized dextrans; aminated, e.g., containing linked aminogroups, polysaccharides or oligosaccharides, linear or branched; carboxylated, carboxymethylated, sulfated or phosphorylated polysaccharides or oligosaccharides, e.g., reacted with derivatives of carbonic, dicarbonic, sulfuric, aminosulfuric, phosphoric acids with resultant linking of carboxylic, aminocarboxylic, carboxymethyl, sulfuric, amino or phosphate groups. Such oligosaccharides may be obtained by chemical alteration of, e.g., dextran, mannan, xylan, pullulan, cellulose, chytosan, agarose, fucoidan, galactan, arabinan, fructan, fucan, chitin, pustulan, levan or pectin. In addition these poly- or oligosachharides may be represented by heteropolymers or homopolymers of monosaccharides such as glucose, galactose, mannose, galactose, deoxyglucose, ribose, deoxyribose, arabinose, fucose, xylose, xylulose, ribulose, polyamidoamine, linear or branched; polyacrylic acid; polyalcohols, e.g., polyvinylalcohol, an polyxylitol, to which carboxylic or aminogroups are chemically linked. The molecular weight of a polyaminoacid is preferably larger than 1000 and smaller than 100000. Polyamino acids with narrow molecular weight (MW) distribution are preferred to those with broad MW distribution. Polyaminoacids are linked with peptide bonds. Polyaminoacids are prepared by chemical synthesis or by recombinant techniques, such as genetic engineering. For additional examples of polymers suitable for use in the present invention see U.S. Pat. Nos. 6,509,323; 6,492,560; 6,468,532; 6,521,736; 6,348,069; 5,871,710; and 6,051,549.

### (ii) Micelles, Reverse Micelles, Liposomes and Microspheres

Amphipathic compounds that contain both hydrophobic and hydrophilic domains are typically organized into vesicular structures such as liposomes, micellar, or reverse micellar structures. Liposomes can contain an aqueous volume that is entirely enclosed by a membrane composed of lipid molecules (usually phospholipids). Micelles and reverse micelles are microscopic vesicles that contain amphipathic molecules but do not contain an aqueous volume that is entirely enclosed by a membrane. In micelles the hydrophilic part of the amphipathic compound is on the outside (on the surface of the vesicle) whereas in reverse micelles the hydrophobic part of the amphipathic compound is on the outside. The reverse micelles thus contain a polar core that can solubilize both water and macromolecules within the inverse micelle. As the volume of the core aqueous pool increases the aqueous environment begins to match the physical and chemical characteristics of bulk water. The resulting inverse micelle can be referred to as a microemulsion of water in oil.

In water, when a sufficient concentration of the two or more components that make up a micelle is present, the components spontaneously aggregate into thermodynamically stable polymeric micelles. The micelle particles assume a microspheroidal shape and possess, in essence, a double layer. The core “layer” forms by virtue of the hydrophobic interactions between, for example, hydrophobic polyesters. Similarly, the surface “layer” forms by virtue of the corresponding hydrophilic interactions of a, for example, hydrophilic polycation with water. A net positive charge will exist around the surface of the micelle, since the hydrophilic segment of the first component is a polycation.

Microemulsification technology improves bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

While all suitable amphiphilic carriers are contemplated, the presently carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

Micelles according to the present invention may comprise biodegradable, biocompatible copolymers, resulting in non-immunogenicity and non-toxicity. In one aspect copolymers disclosed herein degrade into non-toxic, small molecules subject to renal excretion and are inert during the required period of treatment. Degradation may occur via simple hydrolytic and/or enzymatic reaction. Degradation through simple hydrolysis may be predominant when the backbone of a copolymer comprises ester bonds. Enzymatic degradation may become significant in the presence of certain organelles such as lyposomes. The degradation period can be varied from days to months by using polymers of different kinds and molecular weights. In one example, the present invention may use biodegradable polyesters or polypeptides possessing safe and biocompatible degradation pathways. In addition, the highly-branched micellar structure of the present invention may further reduce cytotoxicity since branched polycations such as dendritic polyamidoamines are thought to be less cytotoxic than linear polycations. Accordingly, the advantageous components and structure of polymeric micelles according to the present invention can be appreciated regarding reduced cytotoxicity. For additional examples of micelles, reverse micelles, liposomes, and microspheres suitable for the present invention see U.S. Pat. Nos. 6,338,859, 5,631,018; 6,162,462; 6,475,779; 6,521,211; and 6,443,898.

(iii) Emulsions and Hydrogels

Emulsions as the carrier in the present invention relate to emulsions of an aqueous or an aqueous-organic continuous phase and an organic discontinuous phase, the latter containing an organic solvent which is not miscible with water. Hydrogels are similar and refer to a type of gel in which the disperse phase has combined with water to produce a semisolid material. The emulsions and hydrogels used in the present invention may contain organic compounds from the group of the reaction products of alkylene oxides with compounds capable of being alkylated, such as, for example, fatty alcohols, fatty amines, fatty acids, phenols, alkylphenols, carboximides and resinic acids, preferably balsamic resin and/or abietic acid.

Organic solvents which are not miscible with water include, for example, aliphatic, cycloaliphatic or aromatic hydrocarbons or the acetate-type solvents. Suitable as organic solvents are, preferably, natural, fully- or semisynthetic compounds and, if appropriate, mixtures of these solvents which are fully miscible or soluble with the other compounds of the emulsion in the temperature range of from 20 to 130° C. In one embodiment, suitable solvents are those from the group of the aliphatic, cycloaliphatic or aromatic hydrocarbons which are liquid at room temperature, including oils, such as, for example, mineral oils, paraffins, isoparaffins, fully-synthetic oils such as silicon oils, semisynthetic oils based on, for example, glycerides of unsaturated fatty acids of medium chain length, essential oils, esters of natural or synthetic, saturated or unsaturated fatty acids, for example C₈-C₂₂-fatty acids, C₈-C₁₈-fatty acids, especially preferably methyl esters of rapeseed oil or 2-ethylhexyl laurate, alkylated aromatics and their mixtures, alkylated alcohols, in particular fatty alcohols, linear, primary alcohols obtained by hydroformylation, terpene hydrocarbons and naphtene-type oils, such as, for example, Enerthene. Further organic solvents include those from the group of the acetate-type solvents such as, for example, 1,2-propanediol diacetate, 3-methyl-3-methoxybutyl acetate, ethyl acetate and the like. The solvents can be employed individually or as mixtures with each other.

The continuous aqueous or aqueous-organic phase of the active-agent-containing emulsions or microemulsions according to the present invention contain water, an organic solvent that is soluble or miscible in water, and may also contain at least one natural or synthetic surface-active agent which has a solubility of >10 g/l, in particular >100 g/l in water (d) at 20° C., and, if appropriate, further adjuvants. Organic solvents which are soluble or miscible in water have a solubility in water of >5.0 g/l at 20° C., in particular >15 g/l.

Examples of suitable organic solvents are: aliphatic C₁-C₄-alcohols such as methanol, ethanol, isopropanol, n-propanol, n-butanol, isobutanol or tert-butanol, aliphatic ketones such as acetone, methyl ethyl ketone, methyl isobutyl ketone or diacetone alcohol, polyols, such as ethylene glycol, propylene glycol, butylene glycol, 1,4-butanediol, 1,5-pentanediol, 1,6-hexanediol, diethylene glycol, triethylene glycol, trimethylolpropane, polyethylene glycol or polypropylene glycol with a mean gram-molecular weight of 100 to 4000 g/mol or 200 to 1500 g/mol, or glycerol, monohydroxyethers, such as monohydroxyalkyl ethers or mono-C₁-C₄-alkyl glycol ethers such as ethylene glycol monoethyl ether, ethylene glycol monomethyl ether, diethylene glycol monomethyl ether or diethylene glycolmonoethyl ether, diethylene glycol monobutyl ether, dipropylene glycol monoethyl ether, thiodiglycol, triethylene glycol monomethyl ether or triethylene glycol monoethyl ether, furthermore 2-pyrrolidone, N-methyl-2-pyrrolidone, N-ethyl-pyrrolidone, N-vinylpyrrolidone, 1,3-dimethylimidazolidone, dimethylacetamide and dimethyl formamide.

The amount of the solvents employed in the aqueous continuous phase is in general less than 60% by weight or less than 40% by weight, based on the continuous phase.

Surface-active agents are understood as meaning emulsifiers, wetters, dispersants, antifoams or solubilizers which are soluble or fully soluble, in the aqueous phase. In particular, they can be nonionic, anionic, cationic or amphoteric or of monomeric, oligomeric or polymeric nature. The choice of the surface-active agents is not limited in accordance with the present invention and must be matched with the discontinuous phase to be stabilized with regard to the desired type of emulsion (for example miniemulsion or microemulsion) and the stability of the emulsion, in particular the sedimentation and/or creaming of the disperse phase.

Examples of suitable surface-active agents include the following: a) alkoxylation product which can be obtained by ethylene-oxide-alkoxylation or propylene-oxide-alkoxylation of condensates of phenolic OH-containing aromatics with formaldehyde and NH functional groups; b) inorganic salts which are soluble in water, such as borates, carbonates, silicates, sulfates, sulfites, selenates, chlorides, fluorides, phosphates, nitrates and aluminates of the alkali metals and alkaline earth metals and other metals and also ammonium; c) polymers composed of recurrent succinyl units, in particular polyaspartic acid; d) nonionic or ionically modified compounds form the group of the alkoxylates, alkylolamides, esters, amine oxides and alkyl polyglycosides, including reaction products of alkylene oxides with compounds capable of being alkylated, such as, for example, fatty alcohols, fatty amines, fatty acids, phenols, alkyl phenols, carboximides and resinic acids. These are, for example, ethylene oxide adducts from a class of the reaction products of ethylene oxide with: 1) saturated and/or unsaturated fatty alcohols with 6 to 25 C atoms or 2) alkyl phenols with 4 to 12 C atoms in the alkyl radical or 3) saturated and/or unsaturated fatty amines with 14 to 20 C atoms or 4) saturated and/or unsaturated fatty acids with 14 to 22 C atoms or 5) hydrogenated and/or unhydrogenated resinic acids, or 6) esterification and/or arylation products prepared from natural or modified, optionally hydrogenated castor oil lipid bodies which, if appropriate, are linked by esterification with dicarboxylic acids to give recurrent structural units; e) ionic or nonionic compounds from the group of the reaction products of alkylene oxide with sorbitan ester, oxalkylated acetylene diols and acetylene glycols, and oxalkylated phenols; f) ionic or nonionic polymeric surface-active agents from the group of the homo- and copolymers, graft and graft copolymers and random and linear block copolymers. Examples of such suitable polymeric surface-active agents include polyethylene oxides, polypropylene oxides, polyoxymethylenes, polytrimethylene oxides, polyvinyl methyl ethers, polyethylene imines, polyacrylic acid, polyaryl amides, polymethacrylic acids, polymethacrylamides, poly-N,N-dimethyl-acrylamides, poly-N-isopropyl acrylamides, poly-N-acrylglycinamides, poly-N-methacryl-glycinamides, polyvinyloxazolidones, polyvinylmethyloxazolidones; g) anionic surface-active agents such as, for example, alkyl sulfates, ether sulfates, ether carboxylates, phosphate esters, sulfosuccinate amides, paraffin sulfonates, olefin sulfonates, sarcosinates, isothionates and taurates; h) anionic surface-active agents from the group of what is known as dispersants, in particular condensates which can be obtained by reacting naphthols with alkanols, subjecting alkylene oxide to an addition reaction and at least partially converting the terminal hydroxyl groups into sulfo groups or monoesters of maleic acid, phthalic acid or succinic acid, sulfosuccinic esters, alkylbenzene sulfonates, and salts of the polyacrylic acids, polyethylene sulfonic acids, polystyrene sulfonic acid, polymethacrylic acids, polyphosphoric acids; i) lignin-type compounds, especially lignosulfonates, for example those which have been obtained by the sulfite or Kraft method. They include products which are partially hydrolyzed, oxidized, propoxylated, sulfonated, sulfomethylated or bisulfonated and which are fractionated by known methods, for example according to the molecular weight or the degree of sulfonation. Mixtures of sulfite and Kraft lignosulfonates are also very effective. Suitable are lignosulfonates with a mean molecular weight of greater than about 1,000 to 100,000, a content of active lignosulfonate of at least 80% and, a low content of polyvalent cations. The degree of sulfonation can be varied within wide limits.

In another embodiment, the continuous aqueous phase can also contain, in addition to the abovementioned surface-active agents, water-soluble block or block copolymers; these block or block copolymers include water-soluble block and block copolymers based on ethylene oxide and/or propylene oxide and/or water-soluble block and block copolymers of ethylene oxide and/or propylene oxide on bifunctional amines. Block copolymers based on polystyrene and polyalkylene oxide, poly(meth)acrylates and polyalkylene oxide and also poly(meth)acrylates and poly(meth)acrylic acids are also suitable.

In addition, the continuous aqueous phase can also contain further customary adjuvants such as, for example, water-soluble wetters, antifoams and/or preservatives.

Emulsion types of the present invention which may be mentioned are: macroemulsion: contains droplets >2 μm (microscopic); miniemulsion: droplet diameter 0.1 to 2 μm, turbid; and microemulsion: droplet diameter <0.1 μm; transparent. For additional examples of emulsions and hydrogels suitable for the present invention see U.S. Pat. Nos. 6,458,373 and 6,124,273.

### (iv) Nanoparticles and Microparticles

Examples of nanoparticles and microparticles that can be used as a carrier in the present invention are include porous particles having a mass density less than 1.0 g/cm³, or less than about 0.4 g/cm³. The porous structure permits, for example, deep lung delivery of relatively large diameter therapeutic aerosols, for example greater than 5 μm in mean diameter.

The porous particles preferably are biodegradable and biocompatible, and optionally are capable of biodegrading at a controlled rate for delivery of a drug. The porous particles may be made of any material which is capable of forming a porous particle having a mass density less than about 0.4 g/cm³. Both inorganic and organic materials can be used. For example, ceramics may be used. Other non-polymeric materials may be used which are capable of forming porous particles as defined herein.

The particles may be formed from any biocompatible, and preferably biodegradable polymer, copolymer, or blend, which is capable of forming porous particles having a density less than about 0.4 g/cm³.

Surface eroding polymers such as polyanhydrides may be used to form the porous particles. For example, polyanhydrides such as poly[(p-carboxyphenoxy)-hexane anhydride] (“PCPH”) may be used. Biodegradable polyanhydrides are described, for example, in U.S. Pat. No. 4,857,311.

In another embodiment, bulk eroding polymers such as those based on polyesters including poly(hydroxy acids) can be used. For example, polyglycolic acid (“PGA”) or polylactic acid (“PLA”) or copolymers thereof may be used to form the porous particles, wherein the polyester has incorporated therein a charged or functionalizable group such as an amino acid as described below.

Other polymers include polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses, polysaccharides, and peptides or proteins, or copolymers or blends thereof which are capable of forming porous particles with a mass density less than about 0.4 g/cm³. Polymers may be selected with or modified to have the appropriate stability and degradation rates in vivo for different controlled drug delivery applications.

As another example, the porous particles may be formed from functionalized polyester graft copolymers, as described in Hrkach et al., Macromolecules, 28:4736-4739 (1995); and Hrkach et al., “Poly(L-Lactic acid-co-amino acid) Graft Copolymers: A Class of Functional Biodegradable Biomaterials” in Hydrogel and Biodegradable Polymers for Bioapplications, ACS Symposium Series No. 627, Raphael M. Ottenbrite et al., Eds., American Chemical Society, Chapter 8, pp. 93-101, 1996, the disclosures of which are incorporated herein by reference. The functionalized graft copolymers are copolymers of polyesters, such as poly(glycolic acid) or poly(lactic acid), and another polymer including functionalizable or ionizable groups, such as a poly(amino acid). In another embodiment, comb-like graft copolymers are used which include a linear polyester backbone having amino acids incorporated therein, and poly(amino acid) side chains which extend from the amino acid groups in the polyester backbone. The polyesters may be polymers of α-hydroxy acids such as lactic acid, glycolic acid, hydroxybutyric acid and valeric acid, or derivatives or combinations thereof. The inclusion of ionizable side chains, such as polylysine, in the polymer has been found to enable the formation of more highly porous particles, using techniques for making microparticles known in the art, such as solvent evaporation. Other ionizable groups, such as amino or carboxyl groups, may be incorporated, covalently or noncovalently, into the polymer to enhance porosity. For example, polyaniline could be incorporated into the polymer.

An exemplary polyester graft copolymer, which may be used to form porous polymeric particles is the graft copolymer, poly(lactic acid-co-lysine-graft-lysine) (“PLAL-Lys”), which has a polyester backbone consisting of poly(L-lactic acid-co-Z-L-lysine) (PLAL), and grafted lysine chains PLAL-Lys is a comb-like graft copolymer having a backbone composition, for example, of 98 mol % lactic acid and 2 mol % lysine and poly(lysine) side chains extending from the lysine sites of the backbone.

The use of the poly(lactic acid) copolymer is advantageous since it biodegrades into lactic acid and lysine, which can be processed by the body. The existing backbone lysine groups are used as initiating sites for the growth of poly(amino acid) side chains.

In the synthesis, the graft copolymers may be tailored to optimize different characteristic of the porous particle including: i) interactions between the agent to be delivered and the copolymer to provide stabilization of the agent and retention of activity upon delivery; ii) rate of polymer degradation and, thereby, rate of drug release profiles; iii) surface characteristics and targeting capabilities via chemical modification; and iv) particle porosity. For additional examples of nanoparticles and microparticles suitable for the present invention see U.S. Pat. Nos. 6,447,753 and 6,274,175.

### (v) Solid Surface

In certain embodiments, the carrier used in the present invention may be a solid support, e.g., a polymer bead or a resin, e.g., a Wang resin. Supports can be solids having a degree of rigidity such as silicon, plastic, and the like. Support can also be flexible materials such as plastic or otherwise synthetic materials (such as nylon), materials made of natural polymers (such as cellulose or silk) or derivatives thereof (such as nitrocellulose) and the like. In certain embodiments the support is a porous material which can be rigid or flexible, intermeshed fibers including woven fabrics, and the like. In some embodiments, the solid support is a bead or pellet, which can be porous.

Another option for creating a solid support with reactive sites is to directly derivatize the solid support so that it can be coupled to a compound. The chemistry used to do this can be the same or similar to that used to derivatize controlled pore glass (cpg) beads and polymer beads. Typically, the first step in this process is to create hydroxyl groups (if they do not already exist on the support) or amino groups on the support. If hydroxyl groups exist or are created, they are typically converted to amino groups, for instance by reacting them with gamma-aminopropyl triethoxy silane. MBDs can be added to the amino groups with cyclic acid anhydrides, activated esters, reactions with polymerized alkylene oxides and other methods known to the art.

Another method to increase the reactive surface area of a solid support is to create columnar structures of silicon monoxide, for instance by thermal evaporation of SiOₓ. Another such method is to insert into the reaction cells fabrics, such as non-woven glass or plastic (preferably fiberglass or polypropylene fiber) fabrics and plasma treating the fabric to create reactive sites. Still another method uses spin-on glass, which creates a thin film of nearly stoichiometric SiO₂ from a sil-sesquioxane ladder polymer structure by thermal oxidation. Sol-gel processing creates thin films of glass-like composition from organometallic starting materials by first forming a polymeric organometallic structure in mixed alcohol plus water and then careful drying and baking. When the sol-gel system is dried above the critical temperature and pressure of the solution, an aerogel results. Aerogels have chemical compositions that are similar to glasses (e.g., SiO₂) but have extremely porous microstructures. Their densities are comparably low, in some cases having only about one to about three percent solid composition, the balance being air.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound represented by formula 1:
A-(B)ₙ—C 1
or a pharmaceutically acceptable salt or composition thereof;
wherein
A represents an oligopeptide sequence that is a portion or all of a substrate for an activating protease, wherein said oligopeptide sequence comprises a bond cleavable by said activating protease;
B is independently for each occurrence a D-isomer or L-isomer of a naturally occurring alpha-amino acid;
C represents a chemotherapeutic agent; and
n is an integer in the range of zero to about five.

2. The compound of claim 1, wherein the activating protease is serine protease.

3. The compound of claim 1, wherein the activating protease is selected from the group consisting of fibroblast activation protein (FAP), dipeptidyl-peptidase IV, acylaminoacyl-peptidase, lysosomal Pro-Xaa carboxypeptidase, nucleoporin, lactoferrin, glycosylasparaginase precursor, calpain-2, poliovirus-type picornain 3C, poliovirus-type picornain 2A, parechovirus picornain 3C, adenain, ubiquitinyl hydrolase-L1, ubiquitin-specific peptidase 14, amidophosphoribosyltransferase precursor, autophagin-1, Cezanne deubiquitinating peptidase, otubain-1, CylD protein, UL36 deubiquitinylating peptidase, pepsin A, HIV-1 retropepsin, spumapepsin, presenilin 1, impas 1 peptidase, aminopeptidase N, angiotensin-converting enzyme peptidase unit 1, matrix metallopeptidase-1, neprilysin, carboxypeptidase A1, eupitrilysin, membrane dipeptidase, pappalysin-1, S2P peptidase, AMSH deubiquitinating peptidase, and scytalidoglutamic peptidase.

4. The compound of claim 1, wherein the activating protease is FAP.

5. The compound of claim 1, wherein A is a dipeptide.

6. The compound of claim 1, wherein A is a tripeptide.

7. The compound of claim 1, wherein A is a tetrapeptide.

8. The compound of claim 1, wherein the cleavable bond is carboxy terminal to a dipeptide sequence or subsequence selected from the group consisting of Gly-Pro; Ala-Pro; Trp-Pro; (acetylated)Gly-Pro; (acetylated)Ala-Pro; and (acetylated)Trp-Pro.

9. The compound of claim 1, wherein C is bound to the C-terminus of the A-(B)ₙ oligopeptide sequence.

10. The compound of claim 1, wherein C is an alkylating agent, antimetabolite, anthracycline, plant alkaloid, topoisomerase inhibitors, or an antitumor antibiotic.

11. The compound of claim 1, wherein C is an alkylating agent selected from the group consisting of cyclophosphamide, chlorambucil, estramustine, melphalan, mechlorethamine, ifosfamide, busulfn, treosulfan, thioTEPA, lomustine, carmustine, streptozocin, temozolomide, dacarbazine, cisplatin, carboplatin, oxaliplatin, procarbazine, trofosfamide, and uramustine.

12. The compound of claim 1, wherein C is an antimetabolite selected from the group consisting of methotrexate, pemetrexed, fludarabine cytarabine, 5-fluorouracil, floxuridine, gemcitabine, trimetrexate, mycophenolic acid, tiazofurin, ribavirin, EICAR, doxifluridine, ratitrexed, cytosine arabinoside, mercaptopurine, thioquanine, and capecitabine.

13. The compound of claim 1, wherein C is a plant alkaloid selected from the group consisting of vinblastine, vincristine, vinorelbine, vindesine, etoposide, teniposide, paclitaxel, and docetaxel.

14. The compound of claim 1, wherein C is a topoisomerase inhibitor selected from the group consisting of amsacrin, etoposide, teniposide, 9-aminocamptothecin, crisnatol, topotecan and irinotecan.

15. The compound of claim 1, wherein C is an antitumor antibiotic selected from the group consisting of actinomycin, dactinomycin, doxorubicin, pirarubicin, zorubicin, daunorubicin, epirubicin, idarubicin, mitoxantrone, bleomycin A2, bleomycin B2, peplomycin, mitomycin C, plicamycin, deferoxamine, and hydroxyurea.

16. A method of treating cancer, comprising administering to a subject in need thereof a therapeutically effective amount of a compound of claim 1.

17. The method of claim 16, wherein the subject is a primate, bovine, ovine, equine, porcine, rodent, feline, or canine.

18. The method of claim 16, wherein the subject is a human.

19. The method of claim 16, wherein the cancer is breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, or adenocarcinoma.

20. A method of inhibiting the life-cycle of a cancer cell comprising: contacting the cancer cell with a compound of claim 1.

21. The method of claim 20, wherein the cancer cell is a mammalian cancer cell selected from the group consisting of breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma.

22. The method of claim 20, wherein the cancer cell is a human cancer cell selected from the group consisting of breast cancer, colorectal cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma.
